(19) United States
(12) Patent Application Publication
(10) Pub. No.: US 2004/0024369 A1
(43) Pub. Date: Feb. 5, 2004

(54) Phlebotomy device

(76) Inventors: Christopher Clarke, Edinburg (GB); Jerry Donnan, Edinburgh (GB)

Correspondence Address: BAKER & DANIELS, 111 E. WAYNE STREET, SUITE 800, FORT WAYNE, IN, 46802

(21) Appl. No.: 10/297,774

(22) PCT Filed: Jun. 7, 2001
(86) PCT No.: PCT/GB01/02504

(30) Foreign Application Priority Data
Jun. 13, 2000 (GB) 0014250.5

(51) Int. Cl. A61M 5/00

(52) U.S. Cl. 604/263; 600/576; 128/919

(57) ABSTRACT

A phlebotomy device comprises a barrel provided with a catheter and a needle passing through the catheter, means being provided for automatically retracting the needle from the catheter in response to the insertion of a blood collection vessel into the barrel.

This invention relates to phlebotomy devices.

It is a particular concern in the field of phlebotomy to protect the phlebotomist and others from the risk of accidental needlestick injury and the attendant risk of infection from for instance the AIDS or hepatitis viruses.

Collection of a blood sample or samples from a patient typically involves inserting a needle into a blood vessel and blood drawn into a collection vessel. A catheter may be used in conjunction with an introducer needle to gain access to the blood vessel. A collection vessel may then be connected to the catheter after withdrawal of the introducer needle. After collection of the sample, the catheter may be left in the blood vessel for further sampling or withdrawn from the blood vessel for disposal. It is after withdrawal of the needle assembly that the risk of needlestick injury with potentially infectious blood on the needle is at its greatest.

Current methods of protection frequently rely on isolating the needle tip with a cover or shield after withdrawal of the needle assembly from the patient or by providing the device with some form of needle retraction system which is actuated by the phlebotomist to cause the needle to be retracted into a protective housing. Typically, the needle retraction system uses a stored energy mechanism for providing the retraction effort, requiring manual intervention to initiate the retraction.

Also, while the needle is in situ during sampling, there is a risk that the needle even if inserted correctly may damage the blood vessel, e.g. in the case of an uncooperative or over-active patient.

The present invention addresses the above problems.

According to the present invention there is provided a phlebotomy device comprising a barrel provided with a catheter and a needle passing through the catheter, means being provided for retracting the needle from the catheter in response to the insertion of a blood collection vessel into the barrel.

Preferably the retracting means is operable automatically in response to insertion of the collection vessel.

The force for effecting retraction of the needle may be derived from the force applied to insert the blood collection vessel into the barrel.

The means for retracting the needle may be provided by a tensile element, such as a flexible cord.

The retracted needle may be housed within a chamber of the device.

The arrangement may be such that removal of the blood collection vessel does not displace the needle from its retracted position.

A spike may be provided to pierce a seal of the blood collection vessel.

The needle may be of solid construction.

The arrangement may be such that a mechanical advantage may be obtained whereby the distance travelled by the needle is greater than the distance travelled by the collection vessel during the time the latter is effective to cause needle retraction.

The catheter maybe offset from the central axis of the barrel.

The spike may be centrally located within the barrel and may be in fluid communication with the offset catheter through a radial passageway.

The catheter may be flexible.

There may be an aperture or groove in the needle allowing blood to be visible in the catheter when the needle has penetrated a blood vessel.

The invention will now be described by way of example only with reference to the accompanying drawings, in which:

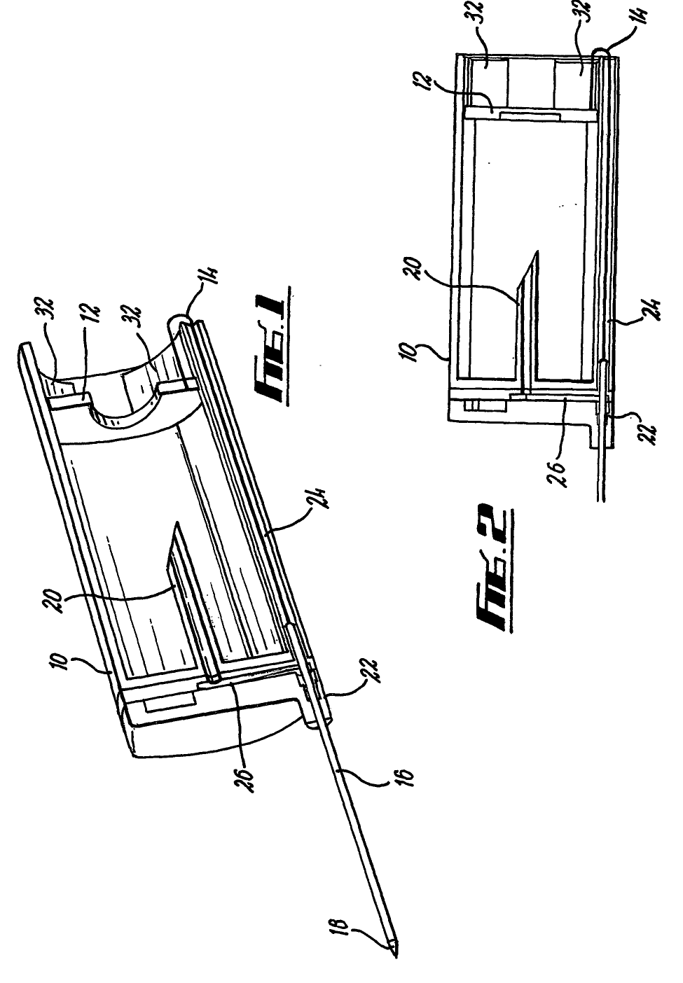

FIG. 1 is a perspective, cross-sectional view of the device in its ready to use state;

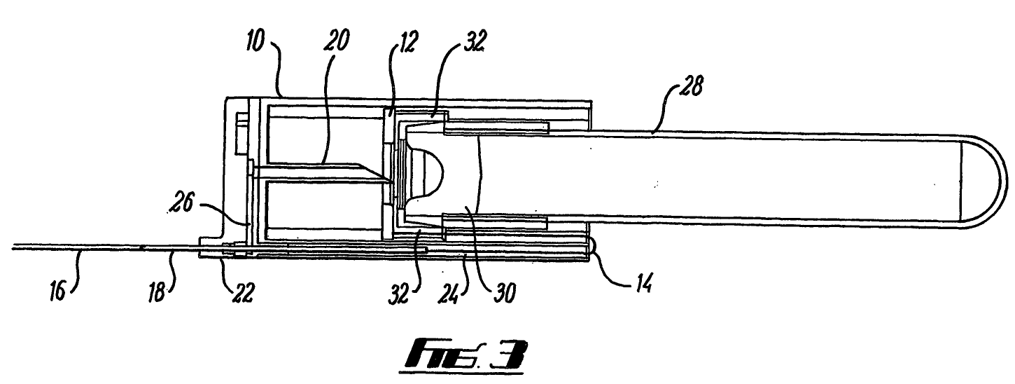

FIG. 2 is a further cross-section of the device in its ready to use state;

FIG. 3 is a cross-section of the device with a collection tube partially inserted;

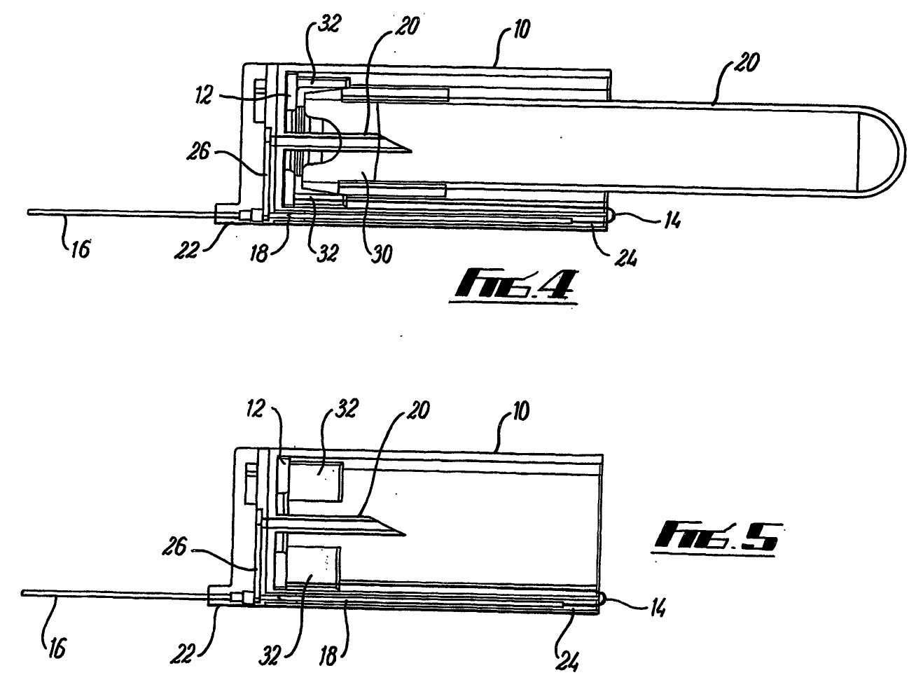

FIG. 4 is a cross-section of the device with the collection tube fully inserted;

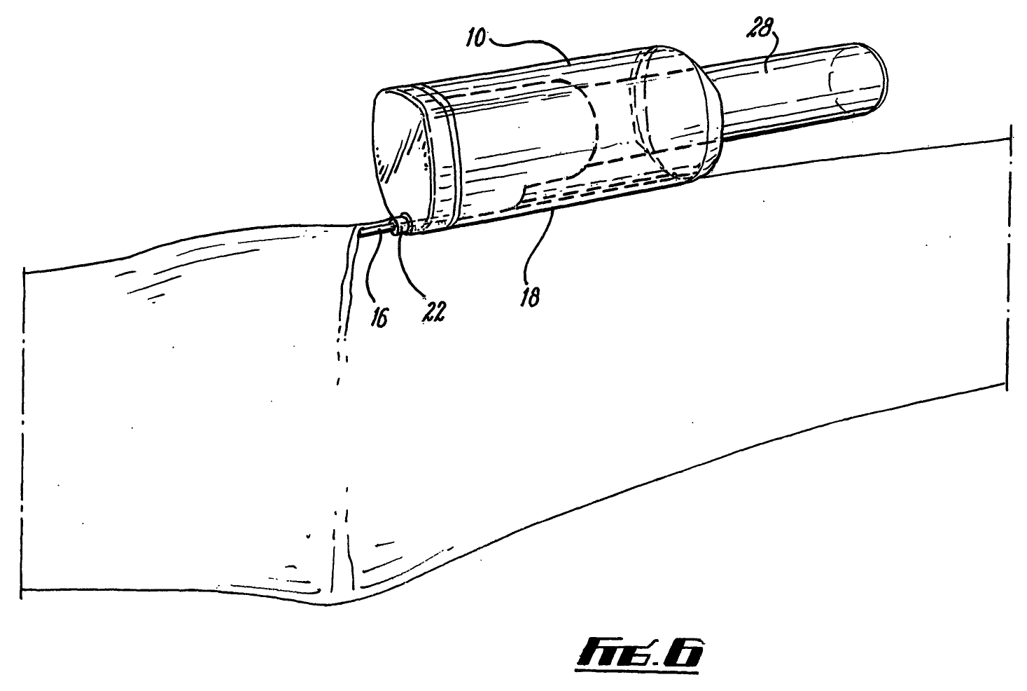

FIG. 5 is a cross-section of the device with the collection tube removed and in readiness for insertion of a further collection tube;

FIG. 6 is an illustration of the device in use on a patient.

Referring to FIGS. 1 to 5 of the drawings, it will be seen that the device comprises a barrel 10, open at one end and closed at the other, which holds a forwardly extending venepuncture needle 18 offset from the central axis of the barrel 10. The needle passes through, and projects beyond, a flexible catheter 16. The catheter 16 is attached to the barrel 10 by means of a projecting part of the barrel 22. By being offset from the centre of the barrel 10, the needle 18 can be inserted into the blood vessel at a shallow angle which allows a shorter needle and catheter length compared to the length that would be required if the needle and catheter were located centrally on the barrel. Once inserted, the device can rest comfortably on the patient, as shown in FIG. 6. The needle 18 may be hollow or solid, a solid needle having the advantage of having a better healing potential because of its more efficient cutting tip, and being safer to use due to the absence of a blood filled cavity as commonly found in hollow needles.

A pusher plate 12 having a central aperture axially aligned with a spike 20 is slidably mounted within the barrel 10. Arcuate guide members 32 support and guide the pusher plate 12 within the barrel 10. The axially located hollow spike 20 within the barrel 10 serves to penetrate the stopper 30 of a collection tube 28 when the latter has been inserted into the barrel 10 to a sufficient extent. A communicating radial passageway 26 provides a fluid path routing the blood from the catheter 16 to the spike 20.

A flexible cord 14 links the pusher plate 12 and the needle 18 via the ventral chamber 24, the cord 14 looping back within the device to provide an actuating means for the retraction of the needle 18 into the ventral chamber 24.

In use, the needle 18 and catheter 16 are inserted into a blood vessel with the initial puncture being made by the needle 18 which projects slightly beyond the catheter 16. A groove or sidehole in the needle 18 may be provided so that blood will be immediately visible within the catheter 16 as an aid to correct insertion, the catheter 16 being made of at least partially transparent material for this purpose. A sealed, evacuated collection tube 28 is then inserted into the barrel 10 and moved forwardly within the barrel 10 with consequent puncture of the collection tube stopper 30 by the spike 20.

As the collection tube 28 moves forwards, the manually applied effort serves to push the pusher plate 12 forwardly thereby causing withdrawal of the needle 18 from the catheter 16 into the ventral chamber 24 by means of the flexible cord 14. Thus, as illustrated in FIG. 4, the needle 18 is withdrawn automatically in the ventral chamber 24 in response to insertion of the collection tube 28 into the barrel 10. The catheter 16 remains in the blood vessel and withdrawal of the needle into the ventral chamber 24 clears the radial passageway 26 to allow fluid communication between the catheter 16 and the spike 20 thereby allowing blood to be withdrawn from the blood vessel into the collection tube 28.

A feature of the invention is that the actuating mechanism operates unidirectionally in that once the needle 18 has been retracted, it is not possible to reverse the action, e.g. by pulling the pusher plate 12 backwards. Also, needle retraction may be directly in response to the act of inserting the collection tube 28 rather than through the release of stored energy by a separate action on the part of the phlebotomist. Nevertheless, we do not exclude the possibility of needle retraction being effected by stored energy means, e.g. stored energy which may derived from the act of inserting the collection vessel and/or which may be released to effect needle retraction without requiring the deliberate intervention of the use.

When sufficient blood has been withdrawn into the collection tube 28, the collection tube 28 is withdrawn from the barrel 10 leaving the pusher plate 12 at the catheter end of the barrel 10 and the needle 18 remaining safely withdrawn within the ventral chamber 24 as illustrated in FIG. 5. The device may then either remain in place with the catheter 16 remaining in the blood vessel ready for further blood collection by insertion of a fresh collection tube or can be removed from the patient and disposed of without the risk of needlestick injury.

The use of a flexible catheter 16 is likely to increase patient comfort and moving the needle 18 from the patient into the ventral chamber 24 at an early stage enhances safety for the phlebotomist by reducing the risk of exposure to the needle, and for the patient by reducing the risk of damage to the blood vessel by the needle.

In the illustrated embodiment, the distance travelled by the needle 18 corresponds to the distance travelled by the collection tube 28. In an alternative embodiment, a gearing factor may be introduced so that the ratio of needle travel to collection tube travel is greater than 1, preferably at least 1.3. Thus, in a modification of the illustrated embodiment, the pusher plate 12 may be omitted and the collection tube 28 arranged to act directly on a flexible portion of the cord or a flexible extension thereof, e.g. a piece of tape, stretched across the interior, for instance along a diameter, of the barrel 10 and connected to the needle 18 via the ventral chamber 24. In this embodiment, the front end of the collection tube 28 will engage that portion or extension of the cord causing needle retraction with a mechanical advantage thereby securing a ratio of needle travel to collection tube travel which is in excess of unity.

In the illustrated embodiment the means for withdrawing the needle 18 into the ventral chamber 24 is provided by a flexible cord 14; however other means are possible—for example a lever or a rack and pinion system which may be suitably designed to ensure that only irreversible movement and/or geared-up travel of the needle is obtained.

In a further embodiment a valve is incorporated into the fluid path to allow for the control of the flow of blood within the device.

In another modification, the catheter 16 is joined to the barrel 10 by means of a catheter hub such as a Luer slip or Luer lock fitment, allowing the catheter 16 to be left in the patient after removel of the barrel 10 following sampling. This would allow later infusion or sampling using the catheter 16.

In a further modification, withdrawal of the needle 18 from the catheter 16 may be modified by the use of a lost motion means whereby insertion of the collection tube 28 does not immediately initiate needle retraction. For example, the rearmost position of the pusher plate 12 may be located inwardly of the barrel 10 so that it is not engaged by the collection tube 28 until the collection tube 28 has been partially inserted, through a predetermined distance, into the barrel 10.

1. A phlebotomy device comprising a barrel provided with a catheter and a needle passing through the catheter, means being provided for retracting the needle from the catheter in response to the insertion of a blood collection vessel into the barrel.

2. A device as claimed in claim 1 in which the needle retracting means operates automatically in response to insertion of the blood collection vessel into the barrel.

3. A device according to claim 1 or 2 in which the arrangement is such that the force for effecting retraction of the needle is derived from the force applied to insert the blood collection vessel into the barrel.

4. A device according to any one of the preceding claims in which the means for retracting the needle is provided by a tensile element, such as a flexible cord.

5. A device according to any one of the preceding claims in which the retracted needle is housed within a chamber of the device.

6. A device according to any one of the preceding claims in which the arrangement is such that removal of the blood collection vessel does not displace the needle from its retracted position.

7. A device according to any one of the preceding claims in which a spike is provided to pierce a seal of the blood collection vessel.

8. A device according to any one of the preceding claims in which the needle is of solid construction.

9. A device according to any one of the preceding claims in which the arrangement is such that a mechanical advantage is obtained whereby the distance travelled by the needle is greater than the distance travelled by the collection vessel during the time the latter is effective to cause needle retraction.

10. A device according to any one of the preceding claims in which the catheter is offset relative to the central axis of the barrel.

11. A device according to any one of the preceding claims in which arrangement is such that the spike is centrally located within the barrel and is in fluid communication with the offset catheter through a radial passageway.

12. A device according to any one of the preceding claims in which the catheter is flexible.

13. A device as claimed in any one of the preceding claims in which needle retraction is irreversible.

14. A device according to any one of the preceding claims in which there is an aperture or groove in the needle allowing blood to be visible in the catheter when the needle has penetrated a blood vessel.

15. A device according to any one of the preceding claims in which the catheter is releasably connected to the barrel.

16. A device according to any one of the preceding claims in which the arrangement is such that there is lost motion between the collection vessel and the needle whereby needle retraction is not initiated until the collection vessel has been partially inserted into the barrel.

17. A phlebotomy device substantially as hereinbefore described with reference to, and as shown in, the accompanying drawings.

18. A phlebotomy device comprising a barrel provided with a catheter and a needle passing through the catheter, means being provided for retracting the needle from the catheter in response to the insertion of a blood collection vessel into the barrel.

19. A device as claimed in claim 1 in which the needle retracting means operates automatically in response to insertion of the blood collection vessel into the barrel.

20. A device as claimed in claim 1 in which the arrangement is such that the force for effecting retraction of the needle is derived from the force applied to insert the blood collection vessel into the barrel.

21. A device as claimed in claim 1 in which the means for retracting the needle is provided by a tensile element, such as a flexible cord.

22. A device as claimed in claim 1 in which the retracted needle is housed within a chamber of the device.

23. A device as claimed in claim 1 in which the arrangement is such that removal of the blood collection vessel does not displace the needle from its retracted position.

24. A device as claimed in claim 1 in which a spike is provided to pierce a seal of the blood collection vessel.

25. A device as claimed in claim 1 in which the needle is of solid construction.

26. A device as claimed in claim 1 in which the arrangement is such that a mechanical advantage is obtained whereby the distance traveled by the needle is greater than the distance traveled by the collection vessel during the time the latter is effective to cause needle retraction.

27. A device as claimed in claim 1 in which the catheter is offset relative to the central axis of the barrel.

28. A device as claimed in claim 1 in which arrangement is such that the spike is centrally located within the barrel and is in fluid communication with the offset catheter through a radial passageway.

29. A device as claimed in claim 1 in which the catheter is flexible.

30. A device as claimed in claim 1 in which needle retraction is irreversible.

31. A device as claimed in claim 1 in which there is an aperture or groove in the needle allowing blood to be visible in the catheter when the needle has penetrated a blood vessel.

32. A device as claimed in claim 1 in which the catheter is releasably connected to the barrel.

33. A device as claimed in claim 1 in which the arrangement is such that there is lost motion between the collection vessel and the needle whereby needle retraction is not initiated until the collection vessel has been partially inserted into the barrel.
